(19) United States
(12) Patent Application Publication
(10) Pub. No.: US 2013/0116520 A1
(43) Pub. Date: May 9, 2013

(54) SINGLE AND MULTI NODE, SEMI-DISPOSABLE WEARABLE MEDICAL ELECTRONIC PATCHES FOR BIO-SIGNAL MONITORING AND ROBUST FEATURE EXTRACTION

(71) Applicants: Masoud Roham, San Diego, CA (US); Alexandros Pantelopoulos, San Diego, CA (US); Meysam Azin, San Diego, CA (US)

(72) Inventors: Masoud Roham, San Diego, CA (US); Alexandros Pantelopoulos, San Diego, CA (US); Meysam Azin, San Diego, CA (US)

(21) Appl. No.: 13/598,005

(22) Filed: Aug. 29, 2012

Related U.S. Application Data
(60) Provisional application No. 61/530,208, filed on Sep. 1, 2011.

(51) Int. Cl. A61B 5/0205

(52) U.S. Cl. CPC A61B 5/0205
USPC 600/324

(57) ABSTRACT

A wireless modular, multi-modal, multi-node patch platform is described. The platform preferably comprises low-cost semi-disposable patch design aiming at unobtrusive ambulatory monitoring of multiple physiological parameters. Owing to its modular design it can be interfaced with various low-power RF communication and data storage technologies, while the data fusion of multi-modal and multi-node features facilitates measurement of several bio-signals from multiple on-body locations for robust feature extraction. Exemplary results of the patch platform are presented which illustrate the capability to extract respiration rate from three different independent metrics, which combined together can give a more robust estimate of the actual respiratory rate.

### PRIORITY CLAIM

This application claims priority to and the benefit of U.S. Provisional Application No. 61/530,208, filed Sep. 1, 2011, entitled “Disclosure-Single And Multi Node, Semi-Disposable Wearable Medical Electronic Patch(es) For Bio-Signal Monitoring And Robust Feature Extraction” (Ref. 921,355-029), which is hereby expressly incorporated herein by reference in its entirety as if fully set forth herein.

### FIELD OF THE INVENTION

The present invention relates to patch based sensors. More particularly, they relate to patches for use on a body for bio-signal monitors.

### BACKGROUND OF THE INVENTION

The United States spends more on health care than any other nation, resulting in health expenditure per capita that exceeded $8,086 in 2009 and more than 75% of the total expenditure in on the health care of people with chronic conditions. A total of 133 million Americans (almost half of adult population) at least one chronic illness, resulting in approximately one-fourth of people with chronic conditions having some daily activity limitations. See, e.g., “The Power of Prevention. Chronic disease . . . the public health challenge of the 21st century. Atlanta Ga.”, National Center for Chronic Disease Prevention and Health Promotion, 2009, and “National Health Expenditures Aggregate, Per Capita Amounts, Percent Distribution, and Average Annual Percent Growth, By Source of Funds: Selected Calendar Years 1960-2009”, Baltimore, Md. Centers for Medicare and Medicaid Services.

An example of the burden of chronic health care in the United States is cardiovascular disease, as it is the leading cause of death for men and women; in this condition, a major cause of the economical burden to the public health is recurrent hospitalization. It has been reported that nonpharmacological strategies, such as the improvement to health care access to the patient, improves survival and reduces hospitalization. See, e.g., E. B. Oberg, A. L. Fitzpatrick, W. E. Lafferty, J. P. LoGerfo, “Secondary Prevention of Myocardial Infarction with Nonpharmacologic Strategies in a Medicaid Cohort”, Prev Chronic Dis, 2009.

As a response to the unmet need to improve accessibility to health care access, other groups have devised wearable physiological monitors, mainly concentrated on cardiovascular activity. In particular, these efforts are centered on the continuous monitoring of the electrocardiogram (ECG) through a single disposable “patch” affixed on the chest of the patient, which measures a pre-established lead of the ECG. The result of these initiatives is relatively expensive and limited in use-case scenarios.

Acute and chronic monitoring of bio-signals is essential for most of medical diagnostic applications. Signals range from body bio-electrical signals (e.g. ECG, EMG, EEG, Fetal ECG (FECG) etc.) and sounds from body organs (e.g. Lung and Heart Sound, etc.) optical images (e.g. Ultrasound, etc.). These signals are traditionally recorded using large size medical equipment's that have cables and leads attached to the patient resting at bed. This approach often requires the patients to visit the hospital to receive monitoring, Described Patch could for Pneumonia, CHF, COPD, SLEEP Apnea detection and differentiation.

State-of-the-art patch-type single use devices that are being developed for bio-signal monitoring applications. These devices are intended for single use in that they are put on the body for the suggested monitoring period (e.g. up to 1 or 2 weeks). During that period they might record saved data in the on-device memory or transmit data based on events. At the end of the monitoring period the device is either thrown away or needs to be returned to a physician for clinical evaluation.

All current systems are single node (there is only one patch operating in the system). As a result, differential bio-signal monitoring from multiple distant points on the body is not possible. Also the current approach does not provide synchronous recording of various bio-signals that are recorded from various parts of the body. In addition to that as there is only a single device, if that device provides unreliable, e.g. noisy, measurements then the collected data are rendered unusable.

Currently synchronization for multi sensor networks is done mostly using RF transmission. Having RF transmission on miniature patches is costly and power- and space-hungry.

Continuous monitoring of the atrial blood gases, especially oxygen saturation (SpO2), has several known benefits in diagnosing diseases like sleep apnea, pneumonia and possibly CHF and COPD. These biometrics are traditionally were measured using large and expensive bench-top equipment in hospitals or clinics.

The current portable devices in the market for pulse oximetry usually work on fingertip. These devices are not suitable for continuous monitoring due to interfering with patients normal and daily activities.

The biggest challenge in continuous photoplethysmography in ambulatory patients is handling different types of artifacts especially motion artifact. Numerous efforts in academia and industry have been dedicated to resolve this issue by improving sensor, circuit and algorithms for optical spectroscopy. Despite these efforts, there is no device in the market capable of continuous and reliable monitoring of arterial blood gases (e.g. SpO2).

Respiration monitoring is crucial in several health monitoring scenarios, especially when monitoring patients suffering from chronic diseases such as COPD and CHF. Detection of respiration rate in a continuous and ambulatory manner is both vital and technically challenging. Different estimators might provide varying results and might be unreliable in the presence of significant noise (such as movement artifacts).

Existing techniques and systems for monitoring respiration make use of a single technique to extract the breathing rate. Such techniques include several methods for ECG derived respiration (EDR). Respiratory Sinus Arrhythmia (RSA) derived respiration, Photoplethysmograph derived respiration, chest-wall movement derived respiration using piezoresistive or piezocapacitive sensors, impedance-based respiratory signals and nasal airflow quantification.

The techniques discussed vary in terms of reliability of measurement. Different techniques might be more reliable under different conditions and context, but there is no single method reliable enough for all cases.

It would, therefore, be beneficial to have a modular platform structure to measure a variety of different physiological and environmental parameters in a form factor that lowers the cost to the end user, and can be applied to various conditions beyond only cardiovascular diseases.

### SUMMARY OF THE INVENTION

A wireless modular, multi-modal, multi-node patch platform is described. The platform preferably comprises low-cost semi-disposable patch design aiming at unobtrusive ambulatory monitoring of multiple physiological parameters. Owing to its modular design it can be interfaced with various low-power RF communication and data storage technologies, while the data fusion of multi-modal and multi-node features facilitates measurement of several bio-signals from multiple on-body locations for robust feature extraction. The patch platform exemplary results in the capability to extract respiration rate from three different independent metrics, which combined together can give a more robust estimate of the actual respiratory rate.

A multi-node patch platform is designed to address limitations of previous approaches using a modular low-cost semi-disposable design. In order to show the system versatility, exemplary systems may measure respiration rate, as this variable is a highly relevant parameter in many chronic conditions such as asthma, congestive heart failure and sleep apnea.

In yet another aspect of the invention, a semi-disposable wearable electronic patch for bio-signal monitoring is provided. The innovation includes single semi-disposable, partially reusable adhesively-wearable (i.e. patch type) device(s) that incorporate various sensing modalities for monitoring of bio-signals. And uses general purpose low cost electrodes. In one aspect, the current invention utilizes a semi-disposable design, whereby the electronics of the device are reusable and only the adhesive part, e.g. the electrodes that come in actual contact with the skin, needs to be replaced after each use. This way, repeated use of this device requires only replacement of the inexpensive electrodes, thus also eliminating concerns regarding infections and also lowering the cost of using the system.

In yet another aspect of the invention, multi-node patch arrays include data fusion for robust feature extraction. Synchronized array of described semi-disposable patches (vs. single patch) that provide recording of several bio-signals from multiple points on the body and fusing them together for robust feature extraction. Recording of multiple channels of the same bio-signal from several on-body locations in a synchronized manner ensures that data are collected in a more robust way, since one or more channels might be distorted at any given time and recording several of them can diminish the issue of data integrity. In addition to that, fusion of features extracted from different spatial channels leads to more robust and accurate bio-signal parameter estimation, as there are different estimates available which can be combined together to yield a more precise estimation of the parameter in question. Finally, synchronized traces of multiple bio-signal channels recorded from on-body distributed patches provide a more complete clinical status of the subject.

In yet another aspect of the invention, body channel communication is provided for synchronizing communication in a multi patch array. Patches in the array become synchronized using the signals that is being sent to body channel, either on-line or offline.

In yet another aspect of the invention, a patched ECG assisted reflective photo plethysmography and pulse oximeter is provided. The innovation includes design and development of a low-cost, semi-disposable, wearable electronic patch for reliable and continuous photoplethysmography for extracting heart-rate, SpO2, SpCO and SpCO2. The extracted feature will be transmitted wirelessly or stored locally on a memory card.

In yet another aspect of the invention, a patch array for fetal heart monitoring with hybrid ultrasound and/or pulse oximetry and FECG Sensin is provided. Patches in the array become synchronized using the signals that is being sent to body channel, either on-line or offline.

In yet another aspect of the invention, patches for multi feature respiration monitoring are provided. A Body Area Network of semi-disposable patches distributed on the body collects a variety of biomedical or context signals and transmits the data to a central node. From each data stream a feature waveform for estimation of the respiration rate is extracted. Multiple respiration estimators are eventually combined together using signal quality indicators to derive a final robust metric of breathing rate.

### BRIEF DESCRIPTION OF DRAWINGS

FIG. 1 shows an illustration of multi-modal, multi-node sensing in patch form factor for robust feature extraction.

FIGS. 2A and 2B show the top and bottom plan views, respectively, of the patch with cover removed, and FIGS. 2C and 2D show the bottom and top plan views, respectively, of the cover.

FIGS. 3A and 3B show the top and bottom plan views, respectively, of the patch, and FIGS. 3C and 3D show the bottom and top plan views, respectively, of the snap-in packaging, docking or charging station.

FIG. 4 shows an exploded perspective view of one implementation of the patch.

FIG. 5 shows a block diagram of a multi-point acquisition system.

FIG. 6 shows a block diagram of the patch demonstrating various sensors and external connectivity. The input connector is shared between electrodes in on-body mode and charging leads in charging mode.

FIG. 7 shows a front end bio-electrical amplifier comprising gain programmability and operation mode detection.

FIG. 8 shows a fabricated patch on a flexible PCB (Top), Flexible patch package (Middle) and disposable electrodes mounted on the patch (Bottom). Circuit components and snap connector for electors are placed in front. Battery and external module (e.g. radio) are in the back.

FIG. 9 shows the real-time ECG and 3-axis accelerometer data visualized on an Android-enabled phone.

FIG. 10 shows the respiration rate extraction using accelerometer (left) and ECG signal with two different techniques (right).

FIG. 11 shows the actual respiration rate and estimated respiration rate from the three different metrics described in FIG. 10.

### DETAILED DESCRIPTION OF THE INVENTION

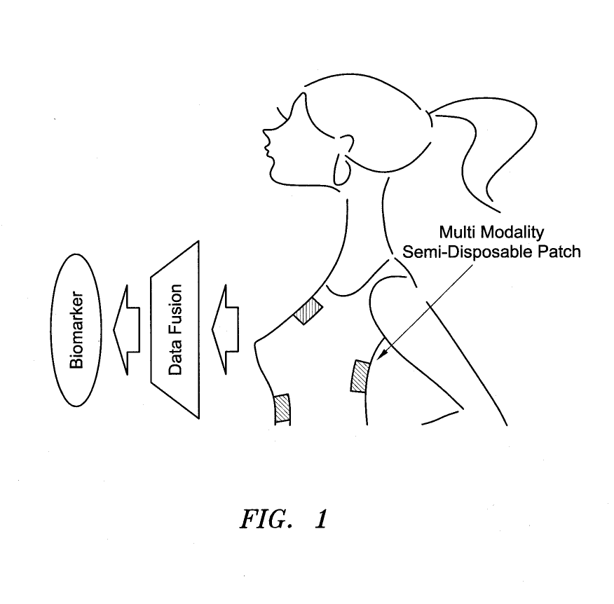

FIG. 1 demonstrates the patch platform concepts. Each patch includes multiple sensing capabilities (e.g. ECG, accelerometer, reflective pulse-oximetry etc.) and packaged in reusable electronics on flexible substrate employing disposable electrodes. The patches are placed in the proper physical position for bio-signal recording. Data fusion algorithm combines the wireless data on either a central node or an external mobile gateway device (e.g. a smartphone or tablet), to cooperatively extract desired biomarker (e.g. respiration).

This semi-disposable, patch design could practically bring down the cost of home monitoring, while collaborative feature extraction increases the reliability of measured biomarkers in such an ambulatory setup. The rest of this section describes the hardware design of fabricated prototype patch including sensing, control, communication and gateway structure.

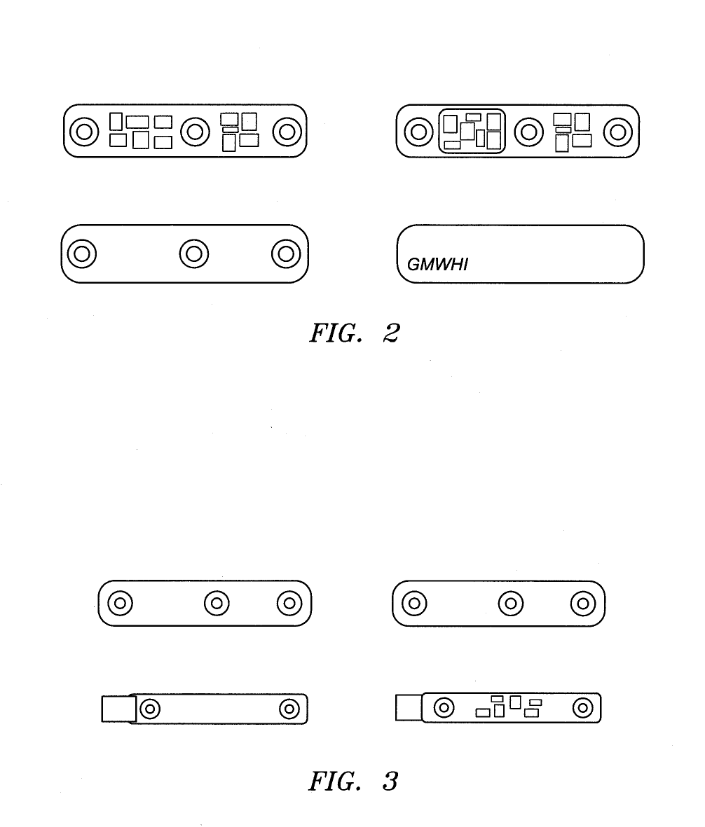

FIGS. 2A (upper left) and 2B (upper right) show the top and bottom plan views, respectively, of the patch with cover removed, and FIGS. 2C (lower left) and 2D (lower right) show the bottom and top plan views, respectively, of the cover. In FIG. 2A, electronic components are located on the surface of the substrate. Multiple snap-in buttons, female in this particular case, are located on the patch. In FIG. 2B, a preferably flexible printed circuit board is disposed on the substrate. The back side of the snap-in buttons are seen, with a connection extending through the substrate. FIG. 2C shows the snap-in button portions on the cover. Optionally, as shown in FIG. 2D, the back of the bottom piece may be blank, or may bear information as to the product, source, or other labeling as desired.

FIGS. 3A (upper left) and 3B (upper right) show the top and bottom plan views, respectively, of the patch, and FIGS. 3C (lower left) and 3D (lower right) show the bottom and top plan views, respectively, of the snap-in packaging, docking or charging station. FIG. 3A shows the top view of the patch with snap-in buttons, in this case male. FIG. 3B shows the electrodes, with an optional bottom adhesive. FIGS. 3C and D show the snap-in packaging, with a docking/charging station connection. The connector, such as a USB connector, is located on the left.

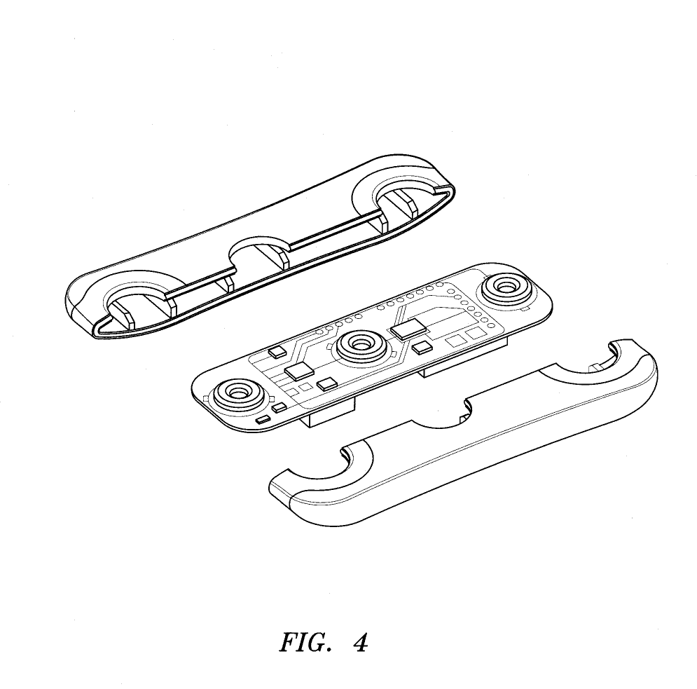

FIG. 4 shows an exploded perspective view of one implementation of the patch. The central substrate supports the electronics, and the snap-in connectors are provided through the substrate.

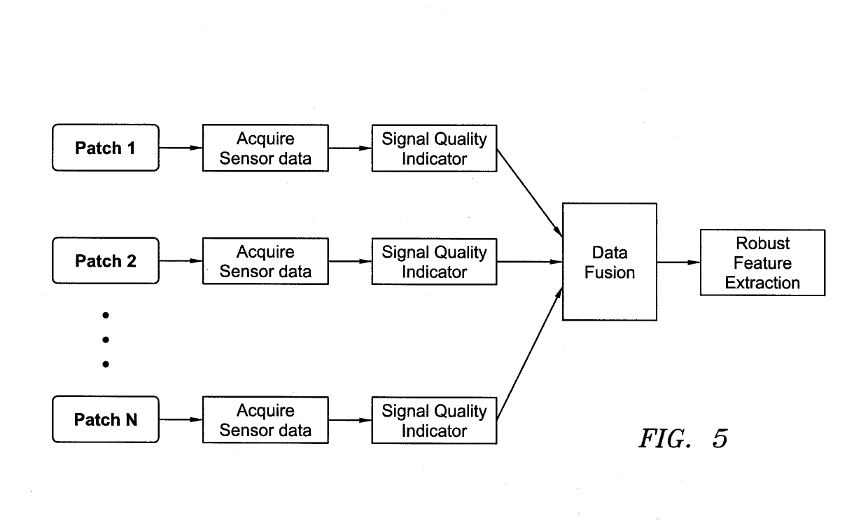

FIG. 5 shows a block diagram of a multi-point acquisition system. The robust feature of extraction is performed after collection of the sensor data. Preprocessing is preferably utilized. Each signal is preferably evaluated individually to derive a signal quality indicator. This parameter along with the signal from each sensor is fused with all other synchronous sensor outputs to derive a robust feature estimation.

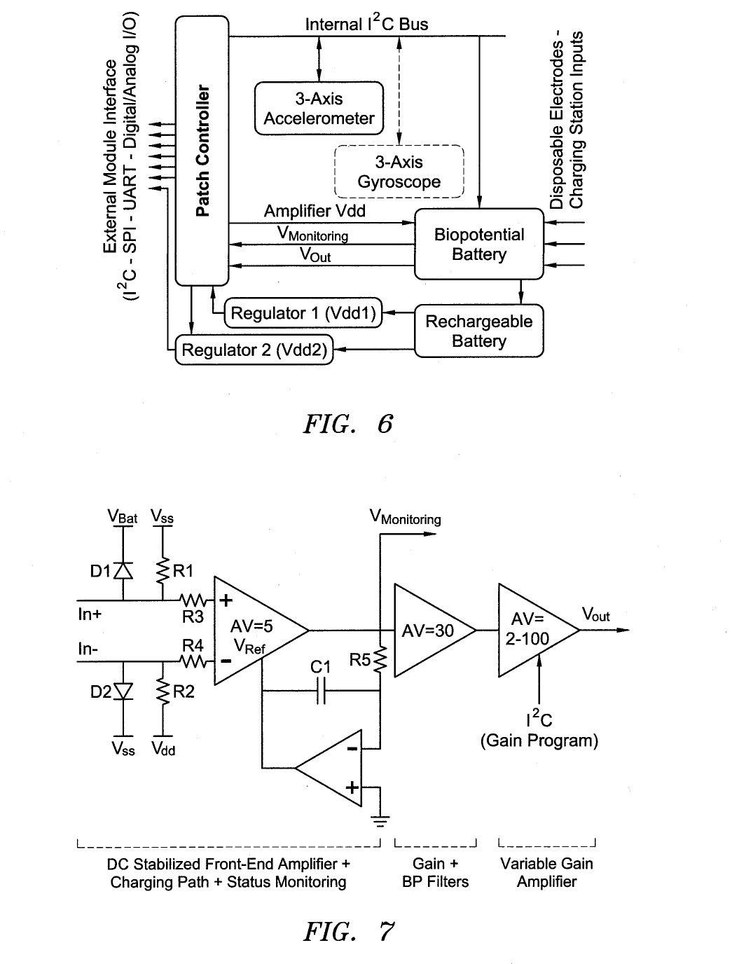

FIG. 6 depicts the overall system diagram of patch hardware. A central microcontroller communicates with configurable biopotential amplifier as well as various sensors on-board using I²C and embedded 10-bit successive approximation analog-digital convertor. The modular design comprises accommodation of external sensors, local data storage and various communication modules. A seven-pin connector provides flexible external peripheral connectivity. It includes a 3.3V regulated voltage, controlled by the patch, and five I/O pins that could be individually configured as general purpose digital or analog I/Os. The pins are also reprogrammable to form either I²C, SPI or UART interfaces.

The system is powered from a 60 mAh 3.7V Lithium Polymer battery regulated by a low-dropout, low-quiescent current 3.3V voltage regulator. During the idle mode, the patch controller periodically wakes-up from the sleep and turns on the amplifier. As it is described later in section II.B, the amplifier automatically identifies the status of patch to be either on-body, on-charge or off-body and turns-on internal and external sensors, accordingly.

The patch may include comprehensive motion detection hardware including a 3-axis MEMS accelerometer as well as a 3-axis gyroscope that is optionally assembled as needed for the use case. It also includes a low power, gain programmable amplifier that accommodates various biopotential signals (i.e. ECG, EMG and EEG).

FIG. 7 depicts the simplified amplifier circuit diagram. During normal operation of the circuit front-end low-leakage diodes (i.e. D₁, D₂) are reverse biased therefore they don't influence the functionality. In order to recharge the battery, charging station applies battery's nominal charging voltage plus twice of the diode forward voltage (i.e. 4.2V+2×V_{F}) to the inputs with a maximum current limited at 60 mA. Therefore, diodes provide charging path.

R₃ and R₄ are introduced to limit the charging current dissipation through ESD protection circuitry of the input instrumentation amplifier within the acceptable range. V_{Monitoring} is monitored by the controller to identify the operation status of the patch. In off-body and charging mode, it saturates around negative and positive rail accordingly. In on-body mode stays within close to the mid supply range (i.e. ground).

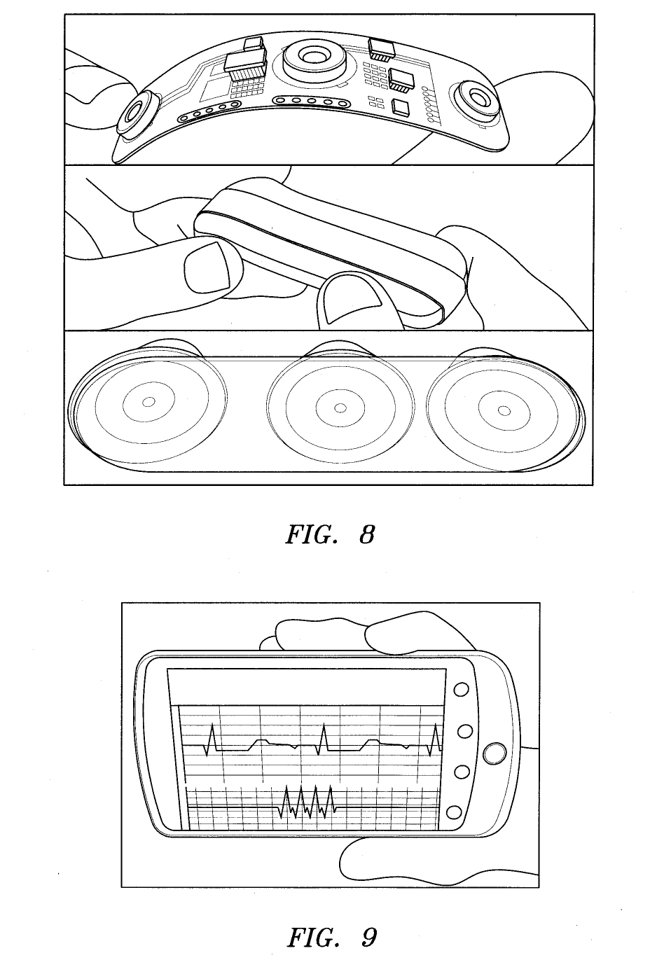

The patch has been designed and fabricated using a three-layer fully flexible polyimide circuit board. FIG. 8 demonstrates the fabricated device and its packaging.

Following the modular design principle the patch can be equipped with different radio technologies depending on the requirements of the given application.

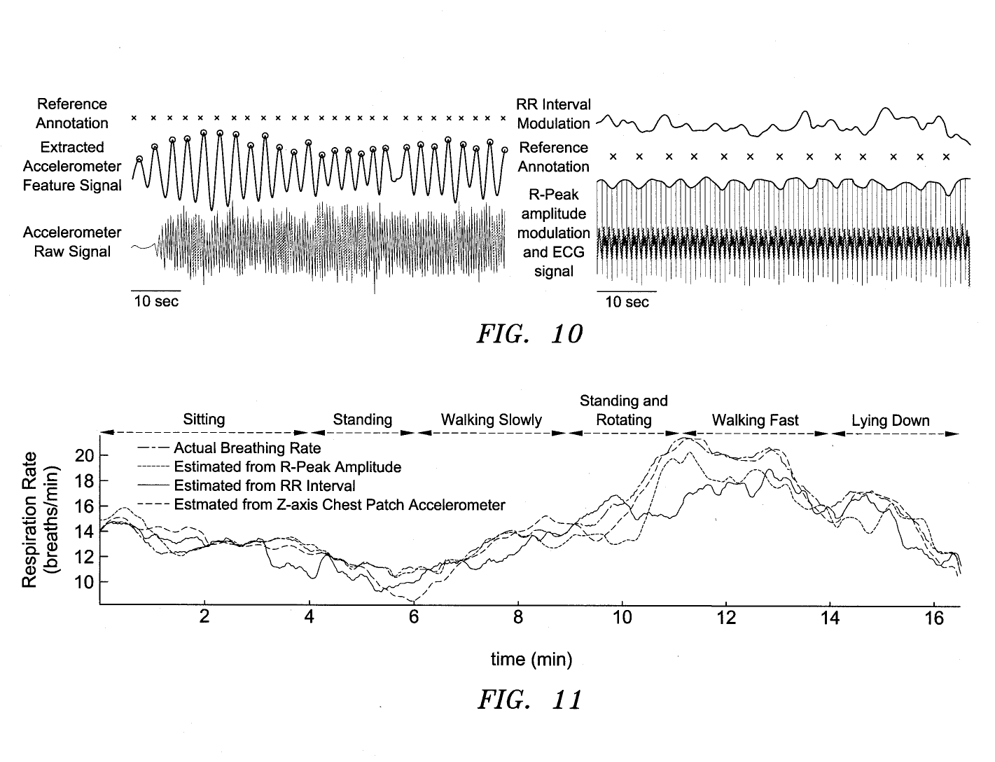

The patch may be implemented with Bluetooth and ANT radio technologies, or any other compatible technology such as Zigbee and Bluetooth Low Energy (BLE) radios. Bluetooth has the major advantage offering high burst data rates and being ubiquitous in consumer electronic devices such as smart-phones and tablet computers. The downside of using Bluetooth radio is high power consumption of the transceiver which in turn limits the operational lifetime of the patch and the fact that the only supported network topology is a Star Network without support for multicast, which makes data synchronization from multiple patches a real challenge. FIG. 10 shows ECG and 3-axis accelerometer data collected from one patch and visualized in real-time on the Android enabled Nexus One.

To address the limitations of standard Bluetooth radio, ANT radio connectivity may be implemented on the patch. ANT has the following competitive advantages over Bluetooth 1) significantly lower transceiver power consumption, see, e.g., T. Vuorela, V-P. Seppa, J. Vanhala, J. Hyttinen, “Wireless Measurement System for Bioimpedance and ECG”, in Proc. of 13^{th} Intl. Conf. on Bioimpedance, 2007, pp. 248-251, 2) smaller software stack, 3) support of complex network topologies, and 4) multi-node synchronization with a beacon-like mechanism. The downside is that although ANT can be found in several sport and wellness devices such as Garmin chest belts, it is not widely available in mobile devices and phones. However, an ANT Android Application Programming Interface (API) was recently released which makes the ANT radio found on some Android enabled phones available to developers. For our tests with ANT-enabled patches have utilized the Sony Ericsson Xperia X8, which includes ANT radio.

Respiration monitoring is a key element in the management of several chronic diseases, such as CHF, Asthma and COPD. Respiration effort can be measured using a variety of methods such as inductive (D. Wu et. al., “A Wearable Respiration Monitoring System Based on Digital Respiratory Inductive Plethysmography”, in Proc. 31^{st} Intl. IEEE EMBC, September 2009, pp. 4844-4847) or impedance plethysmography, chestwall or abdomen movement quantification using piezoresistive or capacitive sensor bands (C. R. Merritt, H. T. Nagle, E. Grant, “Textile-based Capacitive Sensors for Respiration Monitoring”, IEEE Sensors, Vol. 9, pp. 71-78, January 2009) or it can even be indirectly extracted from other bio-signals such as the ECG, J. Boyle et. al., “Automatic Detection of Respiration Rate From Ambulatory Single-Lead ECG”, IEEE Trans. On Inf. Tech. in Biomed., Vol. 13, pp. 890-896. November 2009) and the photoplethysmogram (PPG) (S. G. Flemming, L. Tarassenko, “A Comparison of Signal Processing Techniques for the Extraction of Breathing Rate from the Photoplethysmogram”, Intl. Journal of Biological and Medical Sciences, pp. 232-236, 2007). The system may determine a robust measure of the respiration rate by looking at more than breathing measures. The selected measures are modulation of R-peak amplitude of the ECG, modulation of R-to-R interval of the ECG, and chest wall and abdomen movement quantified with accelerometers.

An 18-minute experiment was conducted on a male volunteer whereby he had 3 patches placed on his body, one over the heart on the chest, one horizontally placed on the abdomen and one more on the same level as the second one only placed on the side of the individual. The user performed various activities during the given time interval, which were: sitting, standing, walking slowly, standing and rotating left and right, walking fast and lying down on his back. The patch on the chest recorded ECG in lead II configuration and 3-axial acceleration and the other two patches captured only the acceleration signals. During the whole test the user was holding an Android enabled phone and was asked to press a button on a custom App at the end of each of his inhalation cycles. These instances were time stamped and were used to evaluate the extracted respiration metrics.

The signal processing and results were as follows. 3 individual respiration metrics were extracted: first, from the modulation of the R peak amplitude and, second, from the modulation of the RR interval of the ECG and third from the frontal plane acceleration signal of the patch placed on the user's abdomen. The first step in extracting signals from ECG (i.e. first and second method) was to detect the R peaks in the recorded signal. The task was performed using the well-known Hamilton-Tomkins algorithm (P. S. Hamilton, W. J. Tompkins, “Quantitative Investigation of QRS Detection Rules using the MIT/BIH Arrhythmia database”, IEEE Trans. Biomedical Engineering, Vol. 33, pp. 1157-1165, 1986.)

After determining the locations of the R peaks, the RR interval and the R-peak amplitude signals were created. Since these two time series contain a small number of samples, they do not lend themselves well to further signal processing, so to increase their comprehensibility they were cubic-spline interpolated. The signals were subsampled to 20 Hz and then band-pass filtered to limit the frequency content in the approximate range of the respiration bandwidth (e.g. 0.1-0.8 Hz or equivalently 6-48 breaths/min). On these resulting waveforms a peak detection algorithm was applied to estimate the time instances of each breath.

Extracting a respiration indicator from accelerometer recordings is a challenge since in case the user is moving the much-lower-amplitude respiration component from the movement of the thorax or the abdomen gets buried in the body motion noise. The use of three different locations was investigated for extracting a respiration metric from accelerometer signals. Testing shows that for the test conditions the abdomen provides the best monitoring location for calculating an estimation of respiratory rate from on-body accelerometers.

In order to isolate the respiratory component present in the accelerometer signal recorded on the abdomen, a stationary biorthogonal wavelet transform using the “bior4.4” MATLAB wavelet was applied. Using this transform, a breathing component (detail signal in the 7^{th} detail scale corresponding to the 0.1-0.5 Hz frequency band) was isolated that is strongly correlated with the manually recorded respiration annotations. This proved to be a robust metric even during the fast walking period of the user.

The performance of the 3 extracted breathing rate metrics is shown in FIG. 11, where the R peak amplitude modulation and the accelerometer derived signal follow very well, the variations of the actual respiration rate. To be more specific, the R peak amplitude derived signal had a 0.94 correlation with the actual respiration annotations, whereas the same number was 0.89 for the RR derived one and 0.99 for the accelerometer-derived metric.

A new multi-modal multi-node scalable patch platform for robust and unobtrusive measurement of a variety of bio-signals is thus provided. Initial results using this new technology for measuring respiration rate using a combination of different breathing metrics extracted from the ECG and accelerometers is provided. Additional sensing modalities may be integrated with the disclosed design to combining these multiple metrics together using a signal quality index for each instance in order to derive a more robust final estimation of the user's respiratory rate.

All publications and patents cited in this specification are herein incorporated by reference as if each individual publication or patent application were specifically and individually indicated to be incorporated by reference. Although the foregoing invention has been described in some detail by way of illustration and example for purposes of clarity and understanding, it may be readily apparent to those of ordinary skill in the art in light of the teachings of this invention that certain changes and modifications may be made thereto without departing from the spirit or scope of the following claims.

We claim:

1. A wireless modular patch for sensing multiple physiologic parameters for a wearer of the patch, comprising:
a plurality of biosensors for detecting and measuring signals corresponding to the physiologic parameters,
a substrate, the substrate supporting the biosensors,
a communications unit, the communications unit being adapted to transmit sensed bio-signals indicative of the physiologic parameters, and
a disposable interface, the interface adapted to contact the wearer, the interface including electrodes which interface with the user.

2. The wireless modular patch for sensing multiple physiologic parameters of claim 1 wherein the disposable interface includes an adhesive surface adapted to interface with the wearer of the patch.

3. The wireless modular patch for sensing multiple physiologic parameters of claim 2 wherein the adhesive is a removable adhesive.

4. The wireless modular patch for sensing multiple physiologic parameters of claim 1 wherein the biosensor includes a respiration sensor.

5. The wireless modular patch for sensing multiple physiologic parameters of claim 1 wherein the biosensor includes an ECG sensor.

6. The wireless modular patch for sensing multiple physiologic parameters of claim 1 wherein the biosensor includes an oxygen sensor.

7. The wireless modular patch for sensing multiple physiologic parameters of claim 6 wherein the oxygen sensor is a photoplethysmography sensor.

8. The wireless modular patch for sensing multiple physiologic parameters of claim 1 wherein one or more patches form a body area network.

9. The wireless modular patch for sensing multiple physiologic parameters of claim further including an accelerometer.

10. The wireless modular patch for sensing multiple physiologic parameters of claim 1 further including an amplifier.
